(19) United States
(12) Patent Application Publication
(10) Pub. No.: US 2010/0114238 A1
(43) Pub. Date: May 6, 2010

(54) INTEGRATION OF FUNCTIONAL ELECTRICAL STIMULATION IN PROSTHETIC SOCKETS, LINERS, AND GARMENTS FOR IMPROVED AMPUTEE CARE AND PERFORMANCE

(76) Inventor: Philip Edward Muccio, Ypsilanti, MI (US)

Correspondence Address: JELIC PATENT SERVICES, LLC, 2922 MARSHALL ST, ANN ARBOR, MI, 48108, US

(21) Appl. No.: 12/608,971

(22) Filed: Oct. 29, 2009

Related U.S. Application Data
(60) Provisional application No. 61/109,813, filed on Oct. 30, 2008.

(51) Int. Cl. A61N 1/36

(52) U.S. Cl. 607/46; 607/48

(57) ABSTRACT

The present disclosure provides a functional electrode stimulation (FES) apparatus for use with prosthetic limbs. FES may provide the benefits of pain management, muscle building, prevention of muscle atrophy, and muscle re-education of residual limb and/or peri-residual limb muscles. The FES apparatus comprises a portable electrical stimulator; means to carry a current between the electrical stimulator and a prosthetic limb liner or socket; a plurality of elastic conductors integrated with the prosthetic limb liner or socket and capable of carrying the current from the means; a plurality of thin planar conductive fabric electrodes capable of carrying the current from the elastic conductors; and a plurality of thin electrodes capable of carrying the current between the thin planar conductive fabric electrodes and the skin of a patient.

### CROSS-REFERENCE TO RELATED APPLICATIONS

This application claims the benefit of U.S. Provisional Application 61/109,813 filed Oct. 30, 2008. The content of this prior application is incorporated by reference in its entirety.

### BACKGROUND OF THE INVENTION

Amputees are a unique subset of individuals with disabilities requiring pain management and improved muscle rehabilitation. The difficulties are that amputees want to wear a prosthetic limb when they are active, but pain can prevent them from wearing a prosthesis, or if they can wear a prosthesis, function is highly compromised. In addition, amputees experience a significant degree of muscle atrophy in the residual limb itself and in the muscles proximal to the amputated limb that diminish various aspects of gait and upper extremity function.

The onset of pain in the amputee can arise from neuromas, phantom pain, degenerative joint disease, compensated gait, undesirable forces and moments on the skeletal system, ill-fitting prosthetic sockets, and other abnormal conditions. Diminished muscle strength can lead to degenerative joint changes as well as poor kinetic and kinematic performance. Because, amputees have missing limb segments, exercise and muscle strengthening can be difficult. Pressure transferred from the interior of the socket to the muscles and soft tissues of the residual limb is known to cause atrophy. Further atrophy may take place from the constriction of muscles inside the prosthetic socket.

### BRIEF SUMMARY OF THE INVENTION

The present disclosure provides a functional electrode stimulation (FES) apparatus for use with prosthetic limbs. FES may provide the benefits of pain management, muscle building, prevention of muscle atrophy, and muscle re-education of residual limb and/or peri-residual limb muscles. The FES apparatus comprises a portable electrical stimulator; means to carry a current between the electrical stimulator and a prosthetic limb liner or socket; a plurality of elastic conductors integrated with the prosthetic limb liner or socket and capable of carrying the current from the means; a plurality of thin planar conductive fabric electrodes capable of carrying the current from the elastic conductors; and a plurality of thin electrodes capable of carrying the current between the thin planar conductive fabric electrodes and the skin of a patient.

Electrical current moves through the apparatus as follows. The portable electrical stimulator generates the electrical current. Current travels through a conductor, typically a thin wire, to the prosthetic limb liner. Then, the positive current travels through a thin fabric conductor, typically silver or another conductive element, to the first thin electrode. The first thin electrode then transfers the current to the skin. Then, the current moves through the body to the second electrode. The return current then conversely moves through thin fabric conductor attached to the second electrode, then to a conductor, then back to the electrical stimulator. Note that there isn't thin fabric conductor located between the two electrodes, since that would create a short circuit. Electrical stimulators typically sends impulses that are biphasic and charge balanced.

The apparatus is designed to avoid adding pressure to the residual limb. The thin fabric conductor is integrated with the liner, minimizing pressure. Also, the electrodes are made of a thin material, typically hydrogel.

### BRIEF DESCRIPTION OF THE DRAWINGS

FIG. 1 shows a cross-section view of the integration of an electrode inside a prosthetic liner.

FIG. 2 shows a prior art application of electrodes on an amputee.

FIG. 3 shows silver fabric electrodes affixed to a liner.

FIG. 4 shows hydrogel adhered to silver fabric electrodes.

FIG. 5 shows the liner 101 being donned by the patient on the residual limb 107.

FIG. 6 shows the liner 101, fully integrated and in place on the residual limb 107.

FIG. 7 shows a pain-free amputee wearing an FES apparatus.

FIG. 8 shows a pain-free amputee walking on an FES apparatus.

### DETAILED DESCRIPTION OF THE INVENTION

FES is known to provide pain relief, reversal of muscle atrophy, and improved circulation, among other benefits. However, using FES on the residual limb inside the socket is not possible with present technology. The present disclosure describes new technology and techniques that enable the integration of electrical stimulation in prosthetic limbs.

Prosthetic sockets are typically custom-made to fit intimately with the amputee limb. Sockets are made from materials that are rigid or semi-rigid. The socket does not normally contact the skin of an amputee. In most cases, an intervening foam or silicone layer overlays the residual limb so that the skin, muscles, and bones of the amputee are cushioned from socket pressures. Contemporary socket design evenly distributes pressure along the surface of the residual limb with some pressure relief over bony prominences.

High pressures inside a socket cannot be tolerated since abnormal pressure beyond that which skin can tolerate will lead to skin breakdown. Conventional electrodes are too thick, adding too much pressure to the socket. Also, conventional electrodes have a short wire connected to a female socket to receive a pin lead. This socket can have an outside dimension of six millimeters or greater, which adds too much pressure to the residual limb. Hence, current technology and techniques are not applicable for FES of amputees, since too much pressure is placed on the residual limb.

The present disclosure provides technology and techniques which overcome the limitations of the current technology and techniques. Instead of conventional electrodes, conductive hydrogel electrodes are used. The hydrogel electrodes are thin, planar, and have sufficient surface area to disperse electrical current across the skin. Also instead of a socket, conductive fabric interwoven with the liner of the prosthetic limb is used. Any pressure added to the residual limb of the amputee is minimal.

The elements of a FES apparatus for use with prosthetic limbs comprise a portable electrical stimulator; means to carry a current between the electrical stimulator and a prosthetic limb liner or socket ; a plurality of elastic conductors integrated with the prosthetic limb liner or socket and capable of carrying the current from the means; a plurality of thin planar conductive fabric electrodes capable of carrying the current from the elastic conductors; and a plurality of thin electrodes capable of carrying the current between the thin planar conductive fabric electrodes and the skin of a patient.

The portable electrical stimulator is capable of generating an electrical current. Typically, a modulated biphasic charge-balanced waveform is used for the current. The modulated biphasic charge-balanced waveform pulses at a predetermined frequency. The current also cycles on and off so that the stimulated muscles do not over-tire.

In one embodiment, an optional heel switch is used to to turn on the stimulation during appropriate times in the gait cycle in which certain muscles should contract. In this embodiment, the prosthetic limb is attached to the bottom of a residual leg. The heel switch is attached to the bottom of the prosthetic limb. The heel switch acts as a trigger for stimulation to induce muscle contractions during swing or stance phase, depending on the clinical outcome desired. For example, the quadriceps muscle extends and stabilizes the knee and prevents it from buckling during stance. A weak quadriceps can be made to contract with electrical stimulation to impart greater knee stability during stance. A typical below the knee amputee may find increased quadriceps activation helpful in terms of improved knee control, reduced muscle atrophy, and pain.

The means to carry a current between the electrical stimulator and a prosthetic limb liner or socket typically comprises a pair of wires. If more than two electrodes are used, there is one wire for each electrode.

The elastic conductor integrated with the prosthetic limb liner and capable of carrying the current is typically silver fabric. The silver fabric forms a fabric mesh with another stretchable fabric. Other metals, such as copper, may be substituted for silver. The conductive fabric is elastic and allows stretching and flexibility without restricting expansion, contraction, flexion, or extension of the residual limb or its muscle expansion. In one embodiment, a stretchy cable consisting of coiled wire encased in an insulating material is also used. The insulating material may be silicone, since it may be chemically bonded with a silicone used to make the prosthetic liner.

The plurality of thin planar conductive fabric electrodes capable of carrying the current from the elastic conductors typically comprise a very thin planar silver fabric or silver alloy fabric. The silver fabric or silver alloy fabric is less than one millimeter. The silver fabric or silver alloy fabric stretches 2-dimensionally to allow the liner to stretch and expand as it is donned.

The plurality of thin electrodes capable of transferring current to the body are typically composed of hydrogel. In one embodiment, the hydrogel is composed of polyethylene glycol which is cross-linked with diacrylate polymers. The hydrogel is electrode is thin so that it doesn't add unnecessary pressure to the residual limb. The hydrogel is replaceable and has adhesive on the side which adheres to the thin planar conductive fabric electrode side. The opposite side is non-adhesive and contacts the patient's skin. The nonadhesive side is designed to be slippery on the skin to allow the electrode to move over the skin without inducing tension and shear forces on the underlying skin. The hydrogel electrode is less than one millimeter thick. In another embodiment, a double-sided adhesive electrode is used.

Typically, two electrodes are used.

The apparatus may be worn without the prosthesis for benefits such as pain management or muscle stimulation.

The elements of the apparatus may be integrated within a prosthetic socket instead of a liner.

In one other embodiment, the flexible liner incorporates electrodes outside of the prosthetic socket.

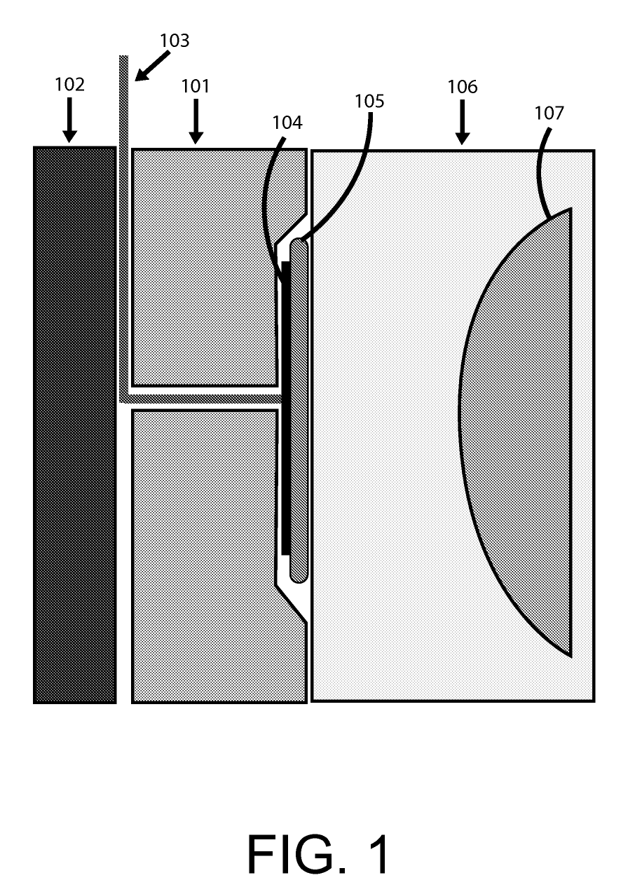

FIG. 1 shows a cross-section view of the integration of an electrode inside a prosthetic liner. The liner 101 is inside a prosthetic socket 102. The liner may be custom-made or pre-made with integrated components in an array that allows the clinician to select the most appropriate point of stimulation for management of pain or stimulation of muscle. Silver fabric (or another metallic conductor) conductor 103 is located between the liner 101 and prosthetic socket 102 and passes through the liner 101 to the silver fabric electrode 104. An adhesive is used to adhere the silver fabric electrode 104 to a conductive hydrogel 105. The conductive hydrogel 105 lies on the surface of the skin 106 and transfers current through the skin 106 to a residual limb 107.

In one embodiment the silver fabric is impregnated with silicone such that the backside become non-conductive and the side facing the patient remains conductive. The silver fabric becomes a more solid material with which to adhere the hydrogel. It also has the advantage of being able to chemically bond with the silicone material of the prosthetic liner.

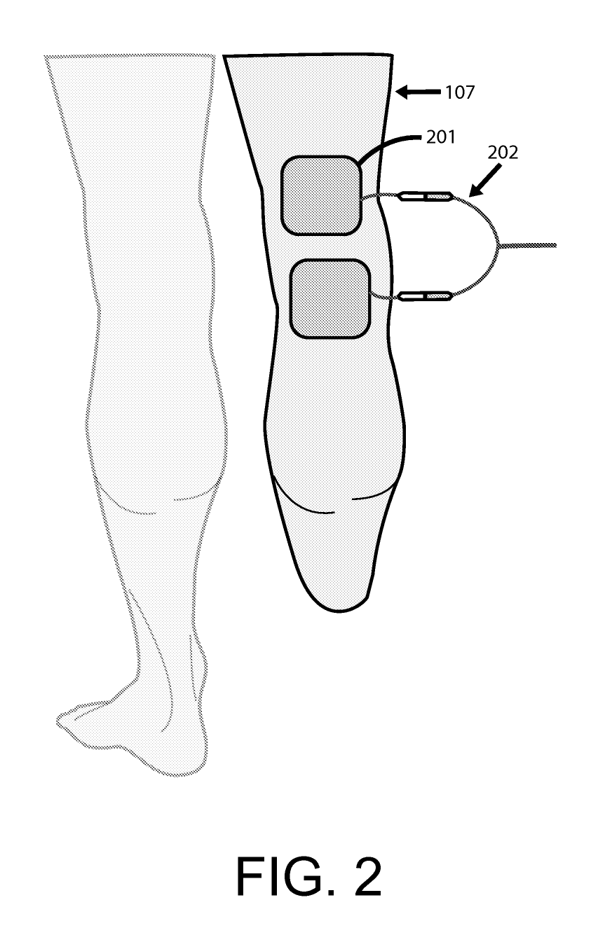

FIG. 2 shows a prior art application of electrodes on an amputee. Two conventional electrodes 201 are positioned on the residual limb 107 of a below-the-knee amputee posteriorly. Each conventional electrode 201 is connected to a lead wire 202. The lead wires cannot be placed in a prosthetic socket since this would add unacceptable pressure.

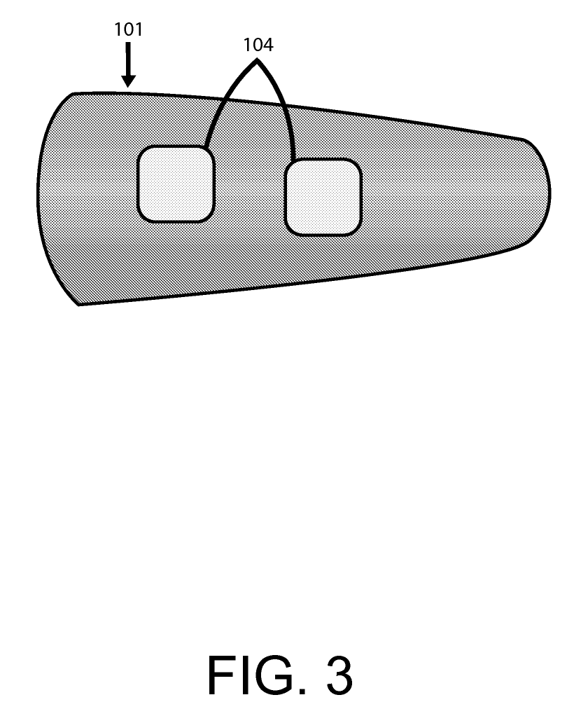

FIG. 3 shows silver fabric electrodes affixed to a liner. The silver fabric electrodes 104 are affixed to the interior of the liner 101 at points specific to the needs of the patient.

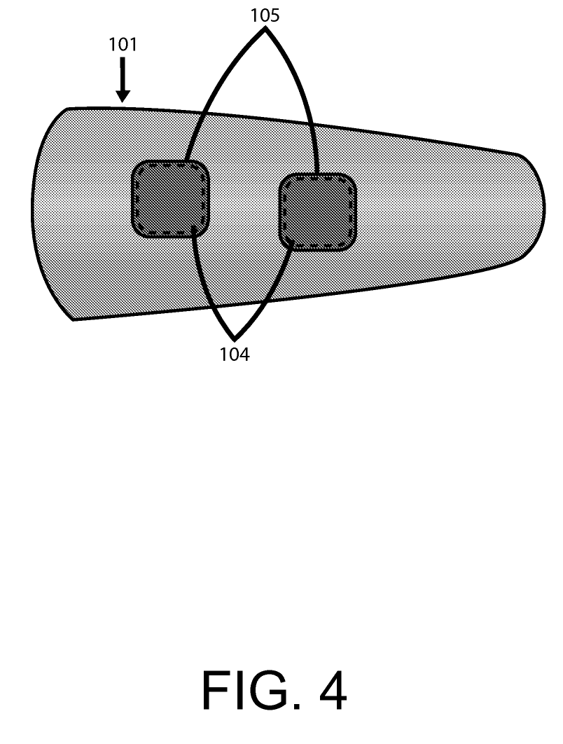

FIG. 4 shows hydrogel adhered to silver fabric electrodes. Hydrogel 105 is adhered to each silver fabric electrode 104 individually. The hydrogel 105 will make contact with the skin 106 when the liner 101 is donned on the residual limb 107.

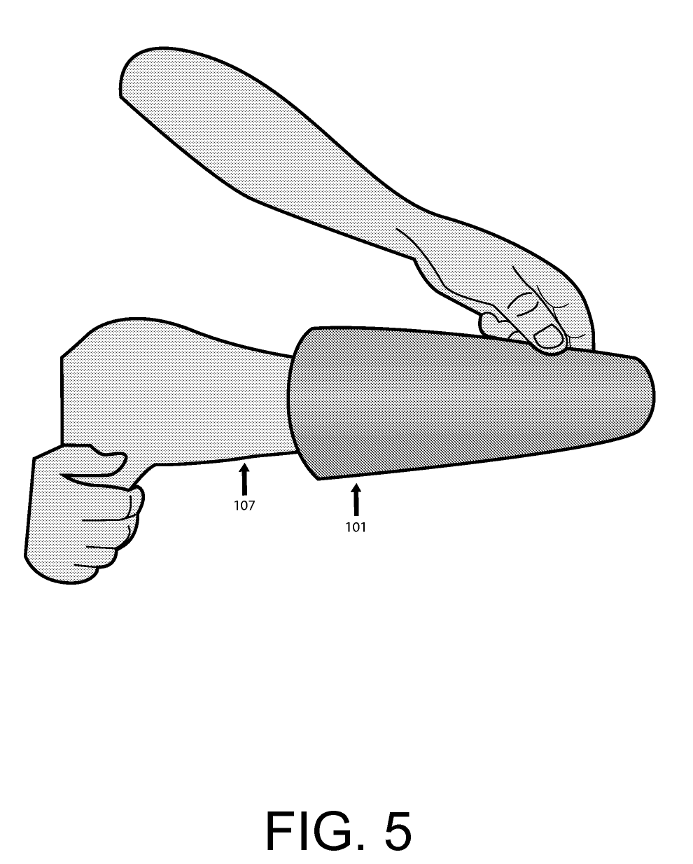

FIG. 5 shows the liner 101 being donned by the patient on the residual limb 107.

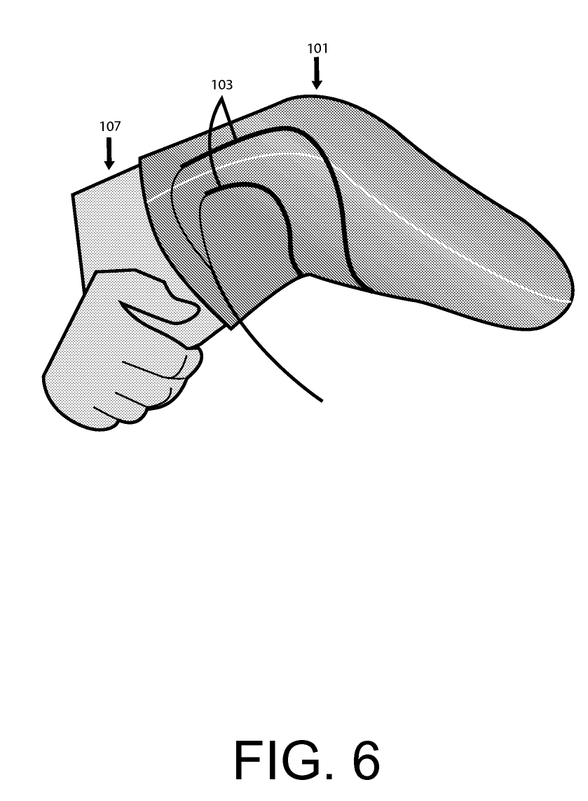

FIG. 6 shows the liner 101, fully integrated and in place on the residual limb 107. Two silver fabric conductors 103 are also shown. The silver fabric conductors 103 are elastic.

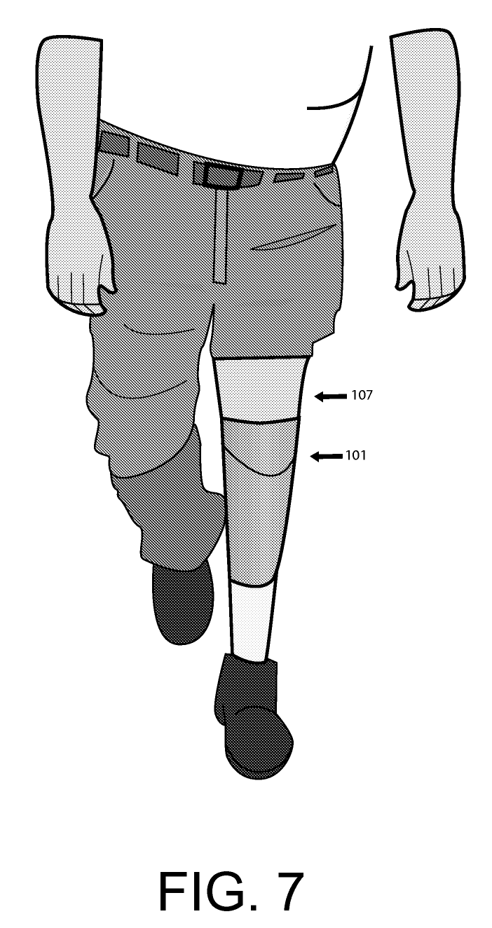

FIG. 7 shows a pain-free amputee wearing an FES apparatus. The amputee is able to bear weight and walk on a prosthesis wearing an integrated liner. The liner does not impose additional pressure to the residual limb since the electrode components and configuration are ultra-thin.

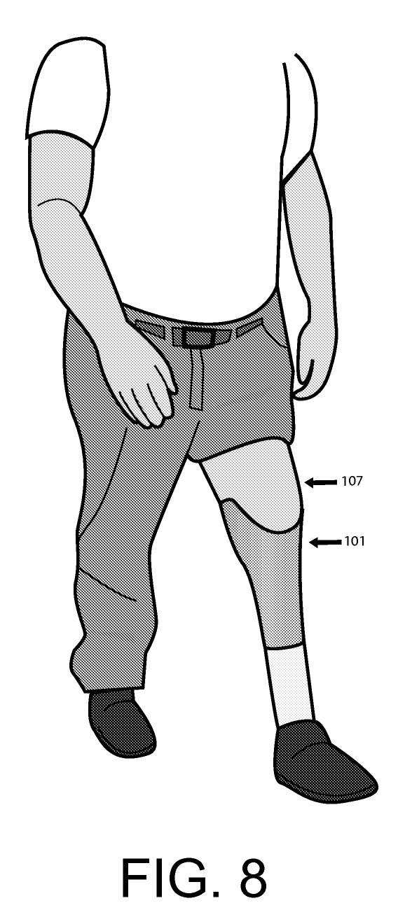

FIG. 8 shows a pain-free amputee walking on an FES apparatus.

While the present invention has been described herein with reference to an embodiment and various alternatives thereto, it should be apparent that the invention is not limited to such embodiments. Rather, many variations would be apparent to persons of skill in the art without departing from the scope and spirit of the invention, as defined herein and in the claims.

I claim:

1. A functional electrode stimulation apparatus for use with prosthetic limbs, the apparatus comprising:
a portable electrical stimulator;
means to carry a current between the electrical stimulator and a prosthetic limb liner;
a plurality of elastic conductors integrated with the prosthetic limb liner and capable of carrying the current from the means;
a plurality of thin planar conductive fabric electrodes capable of carrying the current from the elastic conductors; and
a plurality of thin electrodes capable of carrying the current between the thin planar conductive fabric electrodes and the skin of a patient.

2. The apparatus of claim 1, wherein the elastic conductors comprise a silver fabric or silver alloy fabric.

3. The apparatus of claim 1, wherein the thin planar conductive fabric electrodes comprise a silver fabric or silver alloy fabric.

4. The apparatus of claim 1, wherein the thin electrodes comprise hydrogel.

5. The apparatus of claim 2, wherein the thin planar conductive fabric electrodes comprise a silver fabric or silver alloy fabric.

6. The apparatus of claim 2, wherein the thin electrodes comprise hydrogel.

7. The apparatus of claim 3, wherein the thin electrodes comprise hydrogel.

8. A functional electrode stimulation apparatus for use with prosthetic limbs, the apparatus comprising:
a portable electrical stimulator;
means to carry a current between the electrical stimulator and a prosthetic limb socket;
a plurality of elastic conductors integrated with the prosthetic limb socket and capable of carrying the current from the means;
a plurality of thin planar conductive fabric electrodes capable of carrying the current from the elastic conductors; and
a plurality of thin electrodes capable of carrying the current between the thin planar conductive fabric electrodes and the skin of a patient.

9. The apparatus of claim 1, wherein the elastic conductors comprise a silver fabric or silver alloy fabric.

10. The apparatus of claim 1, wherein the thin planar conductive fabric electrodes comprise a silver fabric or silver alloy fabric.

11. The apparatus of claim 1, wherein the thin electrodes comprise hydrogel.

12. The apparatus of claim 2, wherein the thin planar conductive fabric electrodes comprise a silver fabric or silver alloy fabric.

13. The apparatus of claim 2, wherein the thin electrodes comprise hydrogel.

14. The apparatus of claim 3, wherein the thin electrodes comprise hydrogel.

15. A functional electrode stimulation apparatus for use with a residual limb, the apparatus comprising:
a portable electrical stimulator;
means to carry a current between the electrical stimulator and a residual limb liner;
a plurality of elastic conductors integrated with the residual limb liner and capable of carrying the current from the means;
a plurality of thin planar conductive fabric electrodes capable of carrying the current from the elastic conductors; and
a plurality of thin electrodes capable of carrying the current between the thin planar conductive fabric electrodes and the skin of a patient.
